(19) United States
(12) Patent Application Publication
(10) Pub. No.: US 2023/0190639 A1
(43) Pub. Date: Jun. 22, 2023

(54) FORMULATIONS OF VASOPRESSIN

(71) Applicant: LEIUTIS PHARMACEUTICALS LLP, Hyderabad (IN)

(72) Inventors: Chandrashekhar KOCHERLAKOTA, Secunderabad (IN); Nagaraju BANDA, Hyderabad (IN)

(21) Appl. No.: 18/110,202

(22) Filed: Feb. 15, 2023

Related U.S. Application Data
(63) Continuation of application No. 16/956,631, filed on Jun. 22, 2020, now abandoned.

(30) Foreign Application Priority Data
Dec. 22, 2017 (IN) 201741046283

(51) Int. Cl. A61K 9/00; A61K 38/095; A61K 9/08; A61K 47/02; A61K 47/12; A61K 47/18

(52) U.S. Cl. CPC A61K 9/0019; A61K 38/095; A61K 9/08; A61K 47/02; A61K 47/12; A61K 47/183

(57) ABSTRACT

Provided are novel parenteral formulations of vasopressin, including of vasopressin, stabilizers, solvent and other pharmaceutically acceptable excipients. Also provided are ready to use and/ ready to dilute formulations for parenteral administration. Further provided is a process for preparing formulations of vasopressin for parenteral administration.

### CROSS-REFERENCE TO RELATED APPLICATIONS

This application is a continuation of U.S. application Ser. No. 16/956,631, having a filing date of Jun. 22, 2020, which claims priority to PCT Application No. PCT/IB2018/060498, having a filing date of Dec. 21, 2018, based on IN 201741046283, having a filing date of Dec. 22, 2017, the entire contents all of which are hereby incorporated by reference.

### FIELD OF TECHNOLOGY

Vasopressin is a polypeptide hormone that causes contraction of vascular and other smooth muscles. The vasoconstrictive effects of vasopressin are mediated by vascular Vl receptors. Vasopressin is indicated to increase blood pressure in adults with vasodilatory shock, who remain hypotensive despite fluids and catecholamines. The chemical name of vasopressin is Cyclo (1-6) L-Cysteinyl-L-Tyrosyl-L-Phenylalanyl-L-Glutaminyl-L-Asparaginyl-L-Cysteinyl-L-Prolyl-L-Arginyl-L-Glycinamide.

### BACKGROUND

Vasopressin has been marketed as a therapeutic agent since 1938. Intravenous vasopressin Injection was marketed and sold as an unapproved drug in the United States.

Vasopressin Injection is available under the name Vasostrict® in U.S. It is a sterile, aqueous solution of synthetic arginine vasopressin for intravenous administration. 1 mL solution contains vasopressin 20 units/mL, water for Injection and sodium acetate buffer adjusted to a pH of 3.8. 10 mL solution contains vasopressin 20 units/mL, chlorobutanol 0.5%, water for Injection, and sodium acetate buffer adjusted to a pH of 3.8.

Synthetic vasopressins and derivatives have been disclosed in the following U.S. Pat. No.: 3,371,080 Boissonnas et al., U.S. Pat. No.3,415,805 Siedel et al., U.S. Pat. No.3,418,307 Boissonnas et al., U.S. Pat. No.3,454,549 Boissonnas et al. U.S. Pat. No.3,497,491 Zaoral et al., U.S. Pat. No.4,148,787 Mulder et al.

U.S. Pat. No.5,482,931 to Harris et al., discloses aqueous composition consisting essentially of oxytocin, vasopressin, or its analogues and derivatives; a buffering agent; a quaternary amine preservative or disinfectant and an osmotic pressure-controlling agent.

U.S Pat application 2011/237508 to Jean-Pierre et al., discloses an aqueous formulation comprising of oxytocin, vasopressin or an analogue thereof, a buffer and at least one non-toxic source of divalent metal ions in a concentration of at least 2 mM, having a pH between 3 and 6. The publication specifies that stability can be achieved in buffered solutions only in the presence of divalent metal ions in a concentration of at least 2 mM.

U.S Pat Nos. 9,375,478; 9,687,526; 9,744,209; 9,750,785; 9,744,239 to Matthew et al., disclose pharmaceutical compositions of vasopressin. U.S. Pat. No. 9,375,478 claims unit dosage form consisting of vasopressin, acetate buffer and water, wherein the unit dosage form has a pH of 3.8. U.S. Pat. No. 9,744,239 claims a pharmaceutical composition for intravenous administration consisting of vasopressin, optionally chlorobutanol; acetic acid, acetate, or a combination thereof; wherein the unit dosage form has a pH of 3.5 to 4.1.

All the patents assigned to Matthew et al., disclose the use of acetate buffer. During the patent prosecution, the applicants explained that greatest stability of vasopressin formulations was observed in formulations with acetate buffer with a pH of 3.8.

The inventors of the present invention developed a formulation using alternate stabilizers. These formulations are free of acetate buffer and do not contain divalent metal ions used for stabilizing the formulations of the conventional art.

### SUMMARY

An aspect relates to stable parenteral formulations of vasopressin comprising one or more stabilizers, wherein the formulation is free of acetate buffer.

Another aspect of embodiments of the invention relates to stable parenteral formulations of vasopressin comprising one or more stabilizers, solvents and other pharmaceutically acceptable excipients, wherein the formulation is free of acetate buffer.

Yet another aspect of embodiments of the invention relates to stable parenteral formulations of vasopressin comprising of stabilizers and other pharmaceutically acceptable excipients, wherein the stabilizer comprises (i) one or more buffers and (ii) optionally one or more excipients selected from amino acids and chelating agents.

Yet another aspect of embodiments of the invention relates to ready to use formulations of vasopressin comprising vasopressin in a concentration range of 0.01 units/ml to 2.5 units/ml.

Yet another aspect of embodiments of the invention relates to ready to dilute formulations of vasopressin comprising vasopressin in a concentration range of 2.5units/ml to 100 units/ml.

### DETAILED DESCRIPTION

In the context of embodiments of the invention “vasopressin” refers to pharmaceutically acceptable salts, solvates, hydrates, acids, anhydrous and free base forms thereof, vasopressin.

The term “about” is meant to encompass a range of ±0.5 from the specified value or range.

The term “stable” means the formulations which remain stable, during the entire shelf-life of the composition. The formulation shows an assay of 90 to 110 percent of the original assay value when stored under specified controlled conditions.

The term “stabilizer” refers to an agent which helps in making the formulation stable.

The term “chelating agent” means a compound which helps in improving the stability of the formulation and minimizes the degradation of vasopressin in the formulation.

The term “buffer” means a compound or mixture of compounds that by their presence in the solution resist changes in the pH upon the addition of small quantities of acid or base.

The term “parenteral formulation” encompasses sterile liquid formulations of vasopressin intended for parenteral administration.

Peptides are inherently unstable substances and need to have specific excipients for imparting stability to the formulation. Stabilizers play an important role in maintaining the stability of peptides, particularly those in solution form. The conventional art disclosures describe using acetate buffer or divalent metal ions for stabilizing vasopressin formulations. The pH disclosed in the conventional art in various formulations ranges from about 2.5-4.5. Inventors of the present invention carried out experiments to develop a stable formulation using alternate stabilizers without the use of acetate buffer or divalent metal ions.

One embodiment of the invention comprises parenteral formulations of vasopressin comprising vasopressin, stabilizers and one or more solvents, wherein the formulation is free of acetate buffer.

Vasopressin formulations prepared according to embodiments of the invention were found to be stable over a wide range of pH.

Stabilizers play a significant role in maintaining the stability of vasopressin in liquid formulations. According to embodiments of the present invention, stabilizers include one or more excipients selected from buffers, amino acids and chelating agents. Amino acids are selected from, but not limited to arginine, glycine, alanine, proline, methionine, lysine, leucine, cysteine and isoleucine. Suitable buffers include, but are not limited to Tris, phosphate buffer, citrate buffer, sodium carbonate, sodium bicarbonate, tartarate, benzoate, aspartic acid, ascorbic acid, succinic acid, lactic acid, glutaric acid, malic acid, boric acid, orthophosphoric acid and carbonic acid, alkali or alkaline earth salt of one of these acids. Suitable chelating agents can be selected from, but not limited to DOTA (1,4,7,10-tetraazacyclododecane-1,4,7,10-tetraacetic acid), DTPA (diethylene triamine-N,N,N′,N″,N″-pentaacetate)/pentetic acid, EDTA (Ethylenediamine tetraacetic acid), calcium disodium edetate or their salts. Further other excipients can be selected from lactose, L-aspartic acid, sodium chloride, chlorobutanol.

Suitable solvents include ethanol, glycerine, propylene glycol, polyethylene glycol, water and the like. Water is used as the most common solvent.

The pharmaceutical compositions of embodiments of the present invention may also contain other pharmaceutically acceptable excipients selected from anti-oxidants, preservatives, tonicity modifiers and pH adjusting excipients.

The compositions of embodiments of the present invention can be made as “ready to use” compositions, i.e they can be used without dilution. The concentration of vasopressin in the ready to use formulations ranges from 0.01 units/ml to 2.5 units/ml (i.e 0.00002 mg/ml to 0.0047 mg/ml); additionally in the range of 0.1 units/ml to 1 unit/ml (i.e 0.00019 mg/ml to 0.0019 mg/ml). The compositions of embodiments of the present invention can also be prepared in a “ready to dilute form”, where they need to be diluted with appropriate physiological solutions before administration. The concentration of vasopressin in these formulations ranges from 2.5units/ml to 100 units/ml (i.e 0.0047 mg/ml to 0.18 mg/ml); additionally in the concentration ranges from 5units/ml to 20 units/ml (i.e 0.0094 mg/ml to 0.038 mg/ml); and also in the concentration is in the range of 20 units/ml (0.038 mg/ml).

Inventors carried out experiments to test the stability of vasopressin formulations comprising stabilizers selected from amino acids, buffers and chelating agents. Formulations prepared were tested for stability by subjecting to stress study, at 60° C. for 12 hours.

Formulations prepared using one or more amino acids as stabilizers were tested for stability and the results are given below in table 1.

TABLE 1
Effect of Amino acids on the formulation
Stress study at 60° C. for 12 hours
Vasopressin 0.038 mg/ml

| | | | Total |
|---|---|---|---|
| Amino acids | Chlorobutanol | pH | Impurities |
| L-Cysteine | 5 mg | 3.45 | 4.11 |
| L-Cysteine + IsoLeucine | 5 mg | 3.45 | 5.64 |

The inventors also carried out trials using buffers. Product made with acetate buffer is used as a reference product.

TABLE 2
Effect of buffers on the formulation
Stress study at 60° C. for 12 hours
Vasopressin 0.038 mg/ml

| | | | Total |
|---|---|---|---|
| Buffering excipients | Chlorobutanol | pH | Impurities |
| Orthophosphoric acid | 5 mg | 3.2 | 2.11 |
| Boric acid + Orthophosphoric | 5 mg | 3.1 | 1.39 |
| acid | | | |
| Boric acid + Aspartic acid | 5 mg | 3.1 | 1.41 |
| L-Aspartic Acid | 5 mg | 3.46 | 3.1 |
| Succinic Acid | 5 mg | 3.43 | 2.64 |
| Sodium acetate/Acetic Acid | 5 mg | 3.96 | 3.96 |

Data from tables 1 and 2 shows that (i) amino acids alone will not help in achieving a stable formulation of vasopressin and (ii) buffers help in achieving better impurity profile. Subsequent trials were carried out using a combination of amino acids and buffers and the data is tabulated in table 3.

TABLE 3
Effect of combination of amino acids and buffers on the formulation
Stress study at 60° C. for 12 hours
Vasopressin 0.038 mg/ml

| Buffering excipients; Amino | | | Total |
|---|---|---|---|
| acids | Chlorobutanol | pH | Impurities |
| Succinic Acid + Isoleucine | 5 mg | 3.47 | 1.67 |
| L-Aspartic Acid + Isoleucine | 5 mg | 3.43 | 1.38 |
| L-Ascorbic Acid + Isoleucine | 5 mg | 3.45 | 3.73 |
| L-Aspartic Acid + Arginine | 5 mg | 3.46 | 3.03 |
| Sodium acetate/Acetic Acid | 5 mg | 3.96 | 3.96 |

The level of impurities was considerably higher, for the reference product, made using acetate buffer, when stored at same conditions as embodiments of the invention formulation.

Subsequent trials were carried out to check the stabilizing effect of chelating agents on vasopressin formulation. Various trials were designed, and the samples were stored at an accelerated temperature of 60° C. for 12 hours. The addition of chelating agent was found to further enhance the stability of vasopressin formulations.

TABLE 4
Effect of chelating agent on the formulation

| Ingredients | Qty mg/ml; Vasopressin 0.038 mg/ml strength | | | | | | | | | | | |
|---|---|---|---|---|---|---|---|---|---|---|---|---|
| Orthophosphoric acid | 0.445 | mg | 0.445 | mg | NA | | NA | | NA | | NA | |
| Succinic acid | NA | | NA | | 0.65 | | 0.65 | | NA | | NA | |
| Aspartic acid | NA | | NA | | NA | | NA | | 0.143 | mg | 0.143 | mg |
| Boric acid | NA | | NA | | NA | | NA | | 0.242 | mg | 0.242 | mg |
| DOTA | 0.1 | | NA | | 0.1 | | NA | | 0.1 | | NA | |
| Isoleucine | NA | | NA | | 0.225 | mg | 0.225 | mg | NA | | NA | |
| Chlorobutanol | 5 | mg | 5 | mg | 5 | mg | 5 | mg | 5 | mg | 5 | mg |
| Sodium hydroxide | 0.109 | mg | 0.109 | mg | NA | | NA | | NA | | NA | |
| Water for Injection | Qs 1 | ml | Qs 1 | ml | Qs1 | ml | Qs1 | ml | Qs1 | ml | Qs1 | ml |

| | | | | | | |
|---|---|---|---|---|---|---|
| Total | 1.92 | 2.11 | 1.46 | 1.78 | 1.38 | 1.41 |
| impurities | | | | | | |

One embodiment of the invention provides parenteral formulations of vasopressin comprising 0

a) vasopressin
b) stabilizer comprising
   (i) one or more buffers and
   (ii) one or more excipients selected from amino acids or chelating agents and
c) one or more additional excipients
wherein the formulation is free of acetate buffer

Another embodiment of the invention provides parenteral formulations of vasopressin comprising
a) vasopressin
b) stabilizer comprising
   (i) One or more buffers
   (ii) One or more amino acids and
   (iii) One or more chelating agents, and
c) one or more additional excipients
wherein the formulation is free of acetate buffer.

An embodiment of the invention comprises

| i. | Vasopressin | 0.00002 -0.2% | w/v |
|---|---|---|---|
| ii. | Buffer | q.s to adjust the pH | |
| iii. | Amino acid | 0-10% | w/v |
| iv. | Chelating agent | 0-30% | w/v |
| v. | Water | q.s.t 100% | w/v |
| vi. | Preservative | 0-2% | w/v |

wherein the formulation is free of acetate buffer

The following examples are intended to illustrate embodiments of the invention in more detail and are not to be considered as limiting the scope of embodiments of the invention.

### EXAMPLE 1

| | Quantity | | |
|---|---|---|---|
| Ingredients | | F1 | Reference |

| | | | | |
|---|---|---|---|---|
| Vasopressin | 0.038 | mg | 0.038 | mg |
| Chlorobutanol | 5 | mg | 5 | mg |

| | | | |
|---|---|---|---|
| Succinic Acid | 0.236 | mg | — |

| | | |
|---|---|---|
| Sodium acetate | — | q.s. to adjust |
| | | the pH to 3.8 |

| | | | |
|---|---|---|---|
| Isoleucine | 0.900 | mg | — |
| Water for Injection | Q.s. to 1.0 | mL | |

| | | |
|---|---|---|
| pH | 3.8 | 3.8 |

### Manufacturing Process:

1) Chlorobutanol was added to water for injection and stirred till a clear solution was obtained, at a temperature of about 45-55° C. The solution was cooled.
2) Succinic acid/sodium acetate was added to the above solution followed by isoleucine and stirred till a clear solution was obtained.
3) The above solution was added to vasopressin and stirred.
4) The obtained bulk solution was cooled to 5±3° C.

Formulations prepared according to example 1 were subjected to stability study for a period of 2 months at 25° C./60% RH. Formulation prepared with sodium acetate buffer was taken as reference product. Comparative stability is summarized in table 5.

TABLE 5
Stability data of the formulation prepared according
to example 1 and reference product

| | F1 | | | Reference | | |
|---|---|---|---|---|---|---|
| Parameters | Initial | 1 M | 2 M | Initial | 1 M | 2 M |
| Description | Clear | Clear | Clear | Clear | Clear | Clear |
| pH | 3.68 | 3.62 | 3.81 | 3.74 | 3.68 | 3.80 |
| Total | 0.48 | 1.31 | 1.70 | 0.64 | 1.38 | 1.67 |
| Impurities (% w/w) | | | | | | |
| % Assay | 103.7 | 101.0 | 99.1 | 104.1 | 102.4 | 99.5 |

### EXAMPLE 2

| Ingredients | Quantity | |
|---|---|---|
| Vasopressin | 0.038 | mg |
| L-Aspartic acid | 0.26 | mg |
| L-Isoleucine | 0.9 | mg |
| Water for Injection | QS to 1 | ml |

| | |
|---|---|
| pH | 3.8 |

### Manufacturing Process:

1) L-Aspartic acid and L-Isoleucine were added to water for injection and stirred till a clear solution was obtained.
2) Vasopressin was added to the above solution and stirred.
3) The obtained solution was filled into suitable containers.

### EXAMPLE 3

| Ingredients | Quantity | |
|---|---|---|
| Vasopressin | 0.038 | mg |
| Succinic acid | 0.236 | mg |
| L-Isoleucine | 0.9 | mg |
| Water for Injection | QS to 1 | mL |

| | |
|---|---|
| pH | 3.8 |

### Manufacturing Process:

1) Succinic acid and L-Isoleucine were added to water for injection and stirred till a clear solution was obtained.
2) The obtained solution was added to vasopressin and stirred.

### EXAMPLE 4

| Ingredients | Quantity | |
|---|---|---|
| Vasopressin | 0.038 | mg |
| Chlorobutanol | 5.000 | mg |
| L-Aspartic acid | 0.26 | mg |
| L-Isoleucine | 0.9 | mg |
| Water for Injection | QS to 1 | mL |

| | |
|---|---|
| pH | 3.8 |

### Manufacturing Process:

1) Chlorobutanol was added to water for injection and stirred till a clear solution was obtained, at a temperature of about 45-55° C.
2) The above solution was cooled to room temperature
3) L-Aspartic acid and L-Isoleucine were added to the solution of step 2 and stirred
4) The obtained solution was added to vasopressin and stirred.

### EXAMPLE 5

| Ingredients | Quantity | |
|---|---|---|
| Vasopressin | 0.038 | mg |
| Chlorobutanol | 5.000 | mg |
| Succinic acid | 0.236 | mg |
| L-Isoleucine | 0.900 | mg |
| Water for Injection | QS to 1 | mL |

pH: 3.4

### Manufacturing Process:

1) Chlorobutanol was added to water for injection and stirred till a clear solution was obtained, at a temperature of about 45-50° C.
2) The above solution was cooled to room temperature
3) Succinic acid and L-Isoleucine were added to the solution of step 2 and stirred
4) The above solution was added to vasopressin and stirred.
5) The obtained solution was cooled to 5±3° C.

### EXAMPLE 6

| Ingredients | Quantity | |
|---|---|---|
| Vasopressin | 0.038 | mg |
| Chlorobutanol | 5.000 | mg |
| Succinic acid | 0.236 | mg |
| L-Isoleucine | 0.620 | mg |
| Lactose | 5-15 | mg |
| Water for Injection | QS to 1 | mL |

pH 3.8

### Manufacturing Process:

1) Chlorobutanol was added to water for injection and stirred till a clear solution was obtained, at a temperature of about 45-50° C.
2) The above solution was cooled to room temperature
3) Succinic acid and L-Isoleucine were added to the above solution and stirred till a clear solution was obtained.
4) Lactose was added, followed by the addition of vasopressin to the above solution and stirred

### EXAMPLE 7

| Ingredients | Quantities in mg | | | | |
|---|---|---|---|---|---|
| Vasopressin | 0.038 | 0.038 | 0.038 | 0.038 | 0.038 |
| Chlorobutanol | 5 | 5 | 5 | 5 | 5 |
| Succinic Acid | 2.066 | 2.066 | 2.066 | 2.066 | 2.066 |
| IsoLeucine | 0.900 | 0.900 | 0.900 | 0.900 | 0.900 |
| DOTA | 0.1 | — | — | — | — |
| Calcium disodium | — | 0.1 | — | — | — |
| edetate | | | | | |
| Pentetic acid | — | — | 0.5 | — | — |
| EDTA | — | — | — | 0.1 | — |
| Water for Injection | Q.S to 1.0 mL | Q.S to 1.0 mL | Q.S to 1.0 mL | Q.S to 1.0 mL | Q.S to 1.0 mL |
| pH | 3.2 | 3.2 | 3.2 | 3.2 | 3.2 |

### Manufacturing Process:

1) Chlorobutanol was added to water for injection and stirred till a clear solution was obtained, at a temperature of about 45-50° C.
2) The above solution was cooled to room temperature
3) Succinic acid and L-Isoleucine were added to the above solution and stirred till a clear solution was obtained.
4) DOTA/Calcium disodium edetate/pentetic acid/EDTA was added as per the formula, followed by the addition of vasopressin to the above solution and stirred
5) The solution was cooled to 5±3° C.

### EXAMPLE 8

| Ingredients | Quantity | |
|---|---|---|
| Vasopressin | 0.038 | mg |
| Chlorobutanol | 5 | mg |
| Succinic acid | 4.058 | mg |
| L-Isoleucine | 25.41 | mg |
| Water for Injection | Q.S to 1 | mL |

pH: 4.3

### Manufacturing Process:

1) Chlorobutanol was added to water for injection and stirred till a clear solution was obtained, at a temperature of about 45-55° C.
2) The above solution was cooled to room temperature
3) Succinic acid and L-Isoleucine were added to the above solution and stirred till a clear solution was obtained.
4) The obtained solution was added to vasopressin and stirred.

### EXAMPLE 9

| Ingredients | Quantity | |
|---|---|---|
| Vasopressin | 0.00019 | mg |
| Chlorobutanol | 5.000 | mg |
| Boric acid | 0.252 | mg |
| Ortho phosphoric acid | 0.089 | mg |
| Water for Injection | Q.S to 1 | mL |

pH: 3.1

### Manufacturing Process:

1) Chlorobutanol was added to water for injection and stirred till a clear solution was obtained, at a temperature of about 45-55° C.
2) The above solution was cooled to room temperature
3) Boric acid and orthophosphoric acid were added to the above solution and stirred till a clear solution was obtained.
4) The obtained solution was added to vasopressin and stirred.
5) The solution was cooled to 5±3° C.

### EXAMPLE 10

| Ingredients | Quantity | |
|---|---|---|
| Vasopressin | 0.190 | mg |
| Chlorobutanol | 5.000 | mg |
| Boric acid | 0.252 | mg |
| Ortho phosphoric acid | 0.089 | mg |
| Water for Injection | QS to 1 | mL |

pH: 3.1

### Manufacturing Process:

1) Chlorobutanol was added to water for injection and stirred till a clear solution was obtained, at a temperature of about 45-55° C.
2) The above solution was cooled to room temperature
3) Boric acid and orthophosphoric acid were added to the above solution and stirred till a clear solution was obtained.
4) The obtained solution was added to vasopressin and stirred.
5) The solution was cooled to 5±3° C.

### EXAMPLE 11

| Ingredients | Quantity | |
|---|---|---|
| Vasopressin | 0.00019 | mg |
| Chlorobutanol | 5.000 | mg |
| Succinic acid | 0.650 | mg |
| L-Isoleucine | 0.2250 | mg |
| Water for Injection | QS to 1 | mL |

pH: 3.1

### Manufacturing Process:

1) Chlorobutanol was added to water for injection and stirred till a clear solution was obtained, at a temperature of about 45-55° C.
2) The above solution was cooled to room temperature.
3) Succinic acid and L-Isoleucine were added to the above solution and stirred till a clear solution was obtained.
4) The obtained solution was added to vasopressin and stirred.

### EXAMPLE 12 (ready to use formulations)

Concentration: 1 unit/mL (E.Q. to 0.0019 mg/mL)

| Ingredients | Quantity (mg/ml) | | | |
|---|---|---|---|---|
| Vasopressin | 0.0019 | mg* | 0.0019 | mg* |
| Chlorobutanol | 0.250 | mg | 0.250 | mg |
| Boric acid | 0.0121 | mg | 0.0121 | mg |
| L-Aspartic acid | 0.047 | mg | 0.0016 | mg |
| DOTA | 0.005 | mg | 0.005 | mg |
| Sodium chloride | 9 | mg | 9 | mg |

| | | |
|---|---|---|
| Ultrapure water | QS to 1 mL | QS to 1 ml |
| | (pH 3.1) | (pH 4) |

Concentration: 0.1 unit/mL (E.Q. to 0.00019 mg/mL)

| Ingredients | Quantity (mg/ml) | | | |
|---|---|---|---|---|
| Vasopressin | 0.00019 | mg* | 0.00019 | mg* |
| Chlorobutanol | 0.0250 | mg | 0.250 | mg |
| Boric acid | 0.00121 | mg | 0.0121 | mg |
| L-Aspartic acid | 0.0047 | mg | 0.00016 | mg |
| DOTA | 0.0005 | mg | 0.005 | mg |
| Sodium chloride | 9 | mg | 9 | mg |

| | | |
|---|---|---|
| Ultrapure water | QS to 1 mL | QS to 1 mL |
| | (pH 3.1) | (pH 4) |

Concentration: 0.2 unit/mL (E.Q. to 0.00038 mg/mL)

| | | |
|---|---|---|
| Ingredients | Qty/mL | Qty/mL |

| | | | | |
|---|---|---|---|---|
| Vasopressin | 0.00038 | mg* | 0.00038 | mg* |
| Chlorobutanol | 0.050 | mg | 0.250 | mg |
| Boric acid | 0.00242 | mg | 0.0121 | mg |
| L-Aspartic acid | 0.0094 | mg | 0.00032 | mg |
| DOTA | 0.001 | mg | 0.005 | mg |
| Sodium chloride | 9 | mg | 9 | mg |

| | | |
|---|---|---|
| Ultrapure water | QS to 1 mL | QS to 1 mL |
| | (pH 3.1) | (pH 4.0) |

Q.S: Quantity sufficient;
*Compensated with Assay, Acetic Acid Content and Water Content

Brief Manufacturing Procedure:
1. Transfer 90% of required quantity of ultrapure water into the manufacturing vessel.
2. Add required quantity of chlorobutanol to the above manufacturing vessel and heat the vessel to 50±2° C.
3. Stir the above mixture at 400 RPM to get clear solution at 50±2° C.
4. Cool the above solution to room temperature (25±2° C.).
5. Add required quantity of boric acid to the above manufacturing vessel and stir until clear solution is obtained.
6. Add required quantity of L-Aspartic acid to the above manufacturing vessel and stir until clear solution is obtained.
7. Add required quantity of DOTA to the above manufacturing vessel and stir until clear solution is obtained.
8. Add required quantity of vasopressin API to the above manufacturing vessel and stir until clear solution is obtained.
9. Add required quantity of sodium chloride to the above manufacturing vessel and stir until clear solution is obtained.
10. Make up the final volume of above bulk solution with remaining quantity of ultrapure water and stir well to get clear homogenous solution.
11. Filter the solution using 0.2 μ sterile filter and fill the solution as per the fill volume.
12. Store the filled product below 25° C. or at 2-8° C.

### EXAMPLE 13 (Ready to use Formulations)

| | 0.1 units/ml | | | 0.2 units/ml | | |
|---|---|---|---|---|---|---|
| Ingredients | mg/mL | | % w/v | mg/mL | | % w/v |
| Vasopressin* | 0.00019 | mg | 0.000019 | 0.00038 mg | | 0.000038 |
| | | | | (0.2 units) | | |
| L-Aspartic acid | 0.0672 | mg | 0.00672 | 0.0672 | mg | 0.00672 |
| Boric acid | 0.018 | mg | 0.0018 | 0.018 | mg | 0.0018 |
| Sodium chloride | 9 | mg | 0.9 | 9 | mg | 0.9 |
| Chlorobutanol | 0.025 | mg | 0.0025 | 0.05 | mg | 0.005 |
| Water for Injection | q.s. to 1 | mL | q.s. to 100% | q.s. to 1 | mL | q.s. to 100% |

| | | | | |
|---|---|---|---|---|
| USP Type -1 Clear tubular | 1 | — | 1 | — |
| glass vial with 20 mm neck | | | | |
| (batch# 114A) | | | | |
| Cyclic olefin polymer (COP) | 1 | — | 1 | — |
| vial with 20 mm neck | | | | |
| (Batch# 117A) | | | | |
| ETFE coated Bromobutyl | 1 | — | 1 | — |
| stopper. | | | | |
| Aluminum Tear-off seal | 1 | — | 1 | — |

1. 50% of required quantity of Milli-Q water required for batch was dispensed into a compounding vessel.
2. Dispense required quantity of Chlorobutanol was transferred to the above compounding vessel and heat at 50±2° C. under continuous stirring, till a clear solution was obtained and the solution was then cooled to room temperature.
3. Required quantity of Boric acid was dispensed and added to the above compounding vessel containing Chlorobutanol solution and stirred well to get clear solution.
4. Required quantity of L-Aspartic acid was dispensed and transferred to the above compounding vessel, and stirred well to get a clear solution.
5. Dispense quantity of Sodium chloride was transferred to the above compounding vessel and stirred well to get a clear solution.
6. Dispensed quantity of Vasopressin was transferred to the above compounding vessel and stirred well to get a clear solution.
7. Final volume of the formulation was adjusted the required level by added remaining quantity of Milli-Q water and mixed well to get a clear homogenous solution.
8. pH of the above bulk solution was ensured in the range of 3.30 to 4.0
9. Final bulk solution was filtered through 0.22 μ, PVDF filter.
10. The solutions were then filled in USP Type 1 glass vial with 20 mm neck/COP vials with 20 mm neck and stopped with ETFE coated Bromobutyl stoppers.

The stability was carried out in glass vials and COP vials. The stability data was found to be satisfactory. These unexpected results show that the product can be packaged in COP vials with acceptable stability. This is particularly surprising because there is no product of vasopressin that is packed in COP vials.

TABLE 5
Stability data of Vasopressin RTU injection 0.1 units/mL (0.00019 mg/mL)
in USP Type I clear glass vial:

| | | Storage Condition | | | | | |
|---|---|---|---|---|---|---|---|
| | | Long term storage condition | | | Accelerated storage condition | | |
| | | (2-8° C.) | | | (25° C. ± 2° C./60% ± 5% RH) | | |
| Test | | Time Point | | | | | |
| Parameters | Initial | 1 Month | 2 Months | 3 Months | 1 Month | 2 Months | 3 Months |
| pH | 3.61 | 3.68 | 3.62 | 3.65 | 3.66 | 3.64 | 3.63 |
| Assay (%) | 101.7 | 100.6 | 99.0 | 98.7 | 99.1 | 99.1 | 98.6 |
| Total | 0.27 | 0.31 | 0.65 | 0.96 | 0.70 | 0.96 | 1.38 |
| Impurities | | | | | | | |

TABLE 6
Stability data of Vasopressin RTU injection 0.1 units/mL (0.00019 mg/mL) in COP vial

| | | Storage Condition | | | | | |
|---|---|---|---|---|---|---|---|
| | | Long term storage condition | | | Accelerated storage condition | | |
| | | (2-8° C.) | | | (25° C. ± 2° C./60% ± 5% RH) | | |
| Test | | Time Point | | | | | |
| Parameters | Initial | 1 Month | 2 Months | 3 Months | 1 Month | 2 Months | 3 Months |
| pH | 3.66 | 3.61 | 3.60 | 3.65 | 3.66 | 3.64 | 3.68 |
| Assay (%) | 101.2 | 100.2 | 101.1 | 100.1 | 99.4 | 99.9 | 98.2 |
| Total | 0.26 | 0.50 | 0.93 | 1.00 | 0.87 | 1.56 | 1.73 |
| Impurities | | | | | | | |

Although the invention has been illustrated and described in greater detail with reference to the preferred exemplary embodiments, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1. A parenteral formulation of vasopressin comprising:
a) vasopressin in a concentration from 0.01 units/ml to 2.5 units/ml;
b) stabilizer having:
(i) one or more buffers selected from the group consisting of aspartic acid, succinic acid, boric acid, orthophosphoric acid and combinations thereof, and
(ii) one or more excipients selected from amino acids or chelating agents; and
c) chlorobutanol; and
d) one or more additional excipients,
wherein the parenteral formulation is free of acetate buffer.

2. The parenteral formulation of claim 1, wherein the amino acid is selected from the group consisting of aspartic acid, isoleucine, arginine and combinations thereof.

3. The parenteral formulation of claim 1, wherein chelating agent is selected from the group consisting of DOTA (1,4,7,10-tetraazacyclododecane-1,4,7,10-tetraacetic acid), DTPA (diethylene triamine-N,N,N′,N″,N″-pentaacetate)/pentetic acid, EDTA (Ethylenediamine tetraacetic acid), calcium disodium edetate or their salts and combinations thereof.

4. The parenteral formulation of claim 1, wherein the parenteral formulation is a ready to use solution.

5. The parenteral formulation of claim 1, wherein the parenteral formulation is a ready to dilute solution.

6. The parenteral formulation of claim 1, wherein the parenteral formulation is packed in glass vials or cyclic olefin polymer (COP) vials.

7. A ready to use parenteral formulation of vasopressin comprising:
a) vasopressin in a concentration from 0.01 units/ml to 2.5 units/ml;
b) stabilizer having:
(i) one or more buffers selected from the group consisting of aspartic acid, succinic acid, boric acid, orthophosphoric acid and combinations thereof, and
(ii) one or more excipients selected from amino acids or chelating agents; and
c) chlorobutanol; and
d) one or more additional excipients,
wherein the parenteral formulation is free of acetate buffer.

8. A ready to dilute parenteral formulation of vasopressin comprising:
a) vasopressin in a concentration from 2.5 units/ml to 100 units/ml;
b) stabilizer having:
(i) one or more buffers selected from the group consisting of aspartic acid, succinic acid, boric acid, orthophosphoric acid and combinations thereof, and
(ii) one or more excipients selected from amino acids or chelating agents; and
c) chlorobutanol; and
d) one or more additional excipients,
wherein the parenteral formulation is free of acetate buffer.
